(19) United States
(12) Patent Application Publication
(10) Pub. No.: US 2026/0263090 A1
(43) Pub. Date: Sep. 10, 2026

(54) THROMBECTOMY SYSTEM

(71) Applicant: Boston Scientific Scimed, Inc., Maple Grove, MN (US)

(72) Inventors: Michael William Nagel, Andover, MN (US); Alyssa Madej, Minnetonka, MN (US)

(73) Assignee: Boston Scientific Scimed, Inc., Maple Grove, MN (US)

(21) Appl. No.: 19/555,004

(22) Filed: Mar. 3, 2026

Related U.S. Application Data
(60) Provisional application No. 63/766,612, filed on Mar. 4, 2025.

(51) Int. Cl. A61B 17/22; A61B 17/00; A61B 90/00

(52) U.S. Cl. CPC A61B 17/22; A61B 17/00234; A61B 90/06; A61B 2017/00022; A61B 2017/00199; A61B 2017/00305; A61B 2017/00398; A61B 2017/00544; A61B 2017/00561; A61B 2017/00778; A61B 2017/22079; A61B 2090/064; A61B 2217/005

(57) ABSTRACT

Example thrombectomy systems are disclosed. An example thrombectomy system includes a thrombectomy catheter including a distal end region and a lumen extending therein, a fluid supply tube extending within the lumen of the thrombectomy catheter, wherein the fluid supply tube includes one or more fluid supply jets and a pressure relief member coupled to the thrombectomy catheter, wherein the pressure relief member is configured to control effluent fluid flow within the lumen of the thrombectomy catheter. Further, actuation of the pressure relief member is configured to relieve pressure in the lumen of the thrombectomy catheter.

### CROSS-REFERENCE TO RELATED APPLICATIONS

This application claims the benefit of priority under 35 U.S.C. §119 of U.S. Provisional Application No. 63/766,612, filed Mar. 4, 2025, the entire disclosure of which is hereby incorporated by reference.

### TECHNICAL FIELD

The disclosure is directed to thrombectomy systems. More particularly, the disclosure is directed to a thrombectomy catheter system for improved clot removal.

### BACKGROUND

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be extracted from the vessel through the catheter. Of the known thrombectomy systems and methods, there is an ongoing need to provide alternative configurations of thrombectomy catheters and systems, as well as methods of operating such thrombectomy systems.

### SUMMARY

This disclosure provides design, material, manufacturing method, and use alternatives for medical devices. An example thrombectomy system includes a thrombectomy catheter including a distal end region and a lumen extending therein, a fluid supply tube extending within the lumen of the thrombectomy catheter, wherein the fluid supply tube includes one or more fluid supply jets and a pressure relief member coupled to the thrombectomy catheter, wherein the pressure relief member is configured to control effluent fluid flow within the lumen of the thrombectomy catheter. Further, actuation of the pressure relief member is configured to relieve pressure in the lumen of the thrombectomy catheter.

Alternatively or additionally to any of the examples above, wherein in the pressure relief member includes a pressure relief valve.

Alternatively or additionally to any of the examples above, wherein actuation of the pressure relief valve from a closed configuration to an open configuration is configured to rapidly relieve pressure in the lumen of the distal end region of the thrombectomy catheter.

Alternatively or additionally to any of the examples above, wherein rapidly relieving pressure in the lumen of the distal end region of the thrombectomy catheter generates a vacuum force directed inwardly into the lumen of the thrombectomy catheter.

Alternatively or additionally to any of the examples above, wherein the vacuum force is configured to macerate a thrombus positioned distal to the distal end of the thrombectomy catheter.

Alternatively or additionally to any of the examples above, wherein actuation of the pressure relief member is configured to decrease the pressure within the lumen of the distal end region of the thrombectomy catheter from a peak pressure between 18 psi (124.11 kPa) and 22 psi (151.68 kPa) to a pressure of 0 psi (0 kPa).

Alternatively or additionally to any of the examples above, wherein relieving the pressure from the peak pressure between 18 psi (124.11 kPa) and 22 psi (151.68 kPa) to the pressure of 0 psi (0 kPa) occurs over a time period of 0.075 seconds to 0.4 seconds.

Alternatively or additionally to any of the examples above, wherein actuation of the pressure relief member is configured to decrease the pressure within the lumen of the distal end region of the thrombectomy catheter from a peak pressure between 18 psi (124.11 kPa) and 22 psi (151.68 kPa) to a pressure below 0 psi (0 kPa).

Alternatively or additionally to any of the examples above, wherein the proximal end region of the thrombectomy catheter is coupled to a fluid pump, and wherein the fluid pump is configured to cycle between a downstroke and an upstroke, and wherein actuation of the pressure relief member occurs during the downstroke.

Alternatively or additionally to any of the examples above, wherein the pressure relief member includes a pressure relief valve, and wherein the downstroke occurs between a first time and a second time, and wherein actuation of the pressure relief valve occurs before the second time.

Alternatively or additionally to any of the examples above, wherein actuation of the pressure relief valve opens the pressure relief valve, and wherein the pressure relief valve remains open over a third time period between 0.075 seconds to 0.4 seconds.

Alternatively or additionally to any of the examples above, wherein the pressure relief member includes a roller pump.

Alternatively or additionally to any of the examples above, wherein the roller pump is configured to cycle between an open configuration and a closed configuration, and wherein cycling the roller pump is configured to rapidly relieve pressure in the lumen of the distal end region of the thrombectomy catheter.

Alternatively or additionally to any of the examples above, wherein the roller pump is configured to sense a peak pressure in the lumen of the thrombectomy catheter, and wherein the roller pump is configured to cycle between the open configuration and the closed configuration to rapidly relieve pressure in the lumen of the distal end region of the thrombectomy catheter based on the sensing of the peak pressure.

Another example thrombectomy system includes a processor coupled to a fluid pump and a thrombectomy catheter. Further, the thrombectomy catheter includes a distal end region and a lumen extending therein, a fluid supply tube extending within the lumen of the thrombectomy catheter, wherein the fluid supply tube includes one or more fluid supply jets and a pressure relief member coupled to the processor and the thrombectomy catheter, wherein the pressure relief member is configured to control effluent fluid flow within the lumen of the thrombectomy catheter. Further, actuation of the pressure relief member is configured to relieve pressure in the distal end region of the thrombectomy catheter.

Alternatively or additionally to any of the examples above, wherein in the pressure relief member includes a pressure relief valve configured to rapidly relieve pressure in the lumen of the distal end region of the thrombectomy catheter.

Alternatively or additionally to any of the examples above, wherein the processor is configured to sense a peak pressure value within the lumen of the thrombectomy catheter, and wherein the processor is configured to automatically actuate the pressure relief valve in response to sensing the peak pressure value.

Alternatively or additionally to any of the examples above, wherein the peak pressure value is between 18 psi (124.11 kPa) and 22 psi (151.68 kPa).

Alternatively or additionally to any of the examples above, wherein the processor is configured to actuate the pressure relief valve over a time period between 0.075 seconds to 0.4 seconds.

Another example thrombectomy system includes a thrombectomy catheter including a distal end region and a lumen extending therein. Further, the thrombectomy system includes a fluid supply tube extending within the lumen of the thrombectomy catheter, wherein the fluid supply tube includes one or more fluid supply jets, a pressure relief valve coupled to the thrombectomy catheter, wherein the pressure relief valve is configured to control effluent fluid flow within the lumen of the thrombectomy catheter. Further, the thrombectomy system includes a roller pump coupled to the thrombectomy catheter, wherein the roller pump is configured to control effluent fluid flow within the lumen of the thrombectomy catheter. Further, actuation of the pressure relief valve, the roller pump or both the pressure relief valve and the roller pump is configured to relieve pressure in the lumen of the thrombectomy catheter.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative thrombectomy system;

FIG. 2 is a schematic block diagram of a console management system;

FIG. 3 is a partially exploded view of a portion of the thrombectomy system of FIG. 1;

FIG. 4 is a partially exploded view of a portion of the thrombectomy system of FIG. 1;

FIG. 5 is a cross-sectional view of a distal end region of the thrombectomy catheter of FIG. 1;

FIG. 6 illustrates a thrombectomy catheter being utilized to treat a thrombus inside a body vessel;

FIG. 7 illustrates a thrombectomy catheter being utilized to treat a thrombus inside a body vessel;

FIG. 8 illustrates a thrombectomy catheter being utilized to treat a thrombus inside a body vessel; and

FIG. 9 is a graphical representation of the pressure within a portion of the catheter shown in FIG. 1 over a time period.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Thrombectomy catheters and systems may be used to remove thrombus, plaques, lesions, clots, etc. from veins or arteries. Some thrombectomy catheters may utilize high velocity saline jets in a series to entrain fluid or clot material into and through the shaft of the catheter. Other thrombectomy systems may utilize one or more pressurized saline jets which travel backwards to create a vacuum effect, whereby the vacuum pulls clot material into and through the shaft of the catheter. Accordingly, it may be desirable to configure a thrombectomy system which generates a high vacuum force at the distal end of the catheter, whereby the high vacuum force may generate a high velocity flow of effluent fluid proximally through the catheter to remove thrombus, plaque, blood, etc. from the system. Thrombectomy systems which generate a localized high vacuum force at a distal end of an aspiration catheter are disclosed herein.

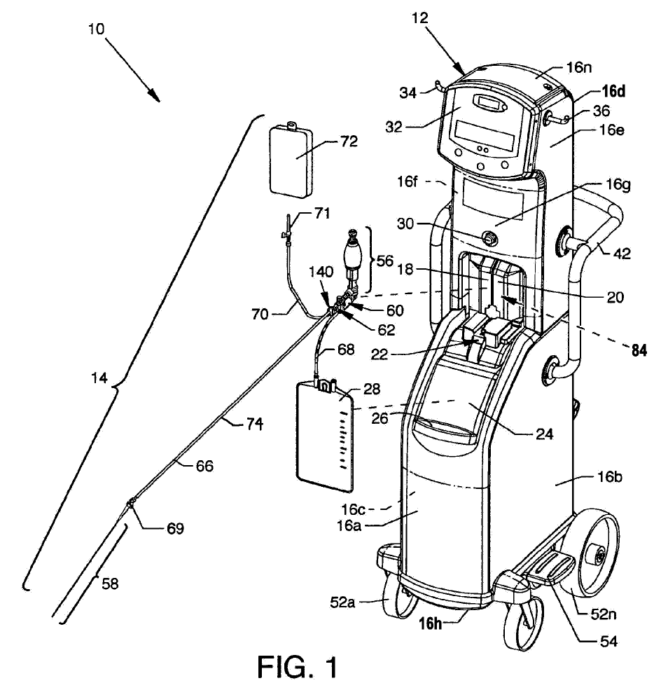

FIG. 1 is a perspective view of an illustrative thrombectomy system 10. The thrombectomy system 10 may include a control console 12 and a pump/catheter assembly 14. In some instances, the pump/catheter assembly 14 may be a single use device in which a new pump/catheter assembly 14 may be used with the console 12 for each medical procedure. Shown on the console 12 are a plurality of removable panels 16a-16i about and along the console 12 enclosing the internal structure of the console 12. An illustrative console 12 is described in commonly assigned U.S. Patent Number 7,935,077, titled THROMBECTOMY CATHETER DEPLOYMENT SYSTEM, the disclosure of which is hereby incorporated by reference.

Centrally located in the console 12 and aligned to the lower region of the panel 16g may be automatically opening doors 18 and 20 which open to expose the interior of the console 12 to provide access to a carriage assembly 22. The carriage assembly 22, which may accommodate components of the pump/catheter assembly 14, as discussed further herein, is shown accessible via opening the closed doors 18 and 20. The console 12 may include a catch basin for collecting fluid leakage from the components of the pump/catheter assembly 14. For example, a removable drip tray 24 is shown located on the front of the console 12 extending from below the carriage assembly 22 toward the panel 16a. Other configurations of catch basins are also contemplated. The drip tray 24 and a removable receptacle 26 may collectively support and accommodate an effluent collection bag, such as effluent collection bag 28 of the pump/catheter assembly 14. In other instances, the console 12 may include a different structure, such as a hook for hanging the effluent collection bag 28. In instances where the carriage assembly 22 is movable, a carriage assembly activation switch 30 may be provided with the console 12, such as located on panel 16g, to selectively position the carriage assembly 22 inwardly or outwardly. A user interface 32, including memory capabilities, may be provided with the console 12, such as located at the upper region of the console 12 between the upper regions of the upper side panels 16e and 16f. Saline bag hooks 34 and 36 may extend through the panels 16e and 16f to hang saline bags therefrom. The console 12 may include a handle 42 as well as a plurality of wheels 52a, 52b and brake pedals 54 for wheel lockage to assist in maneuvering the console 12 by medical personnel.

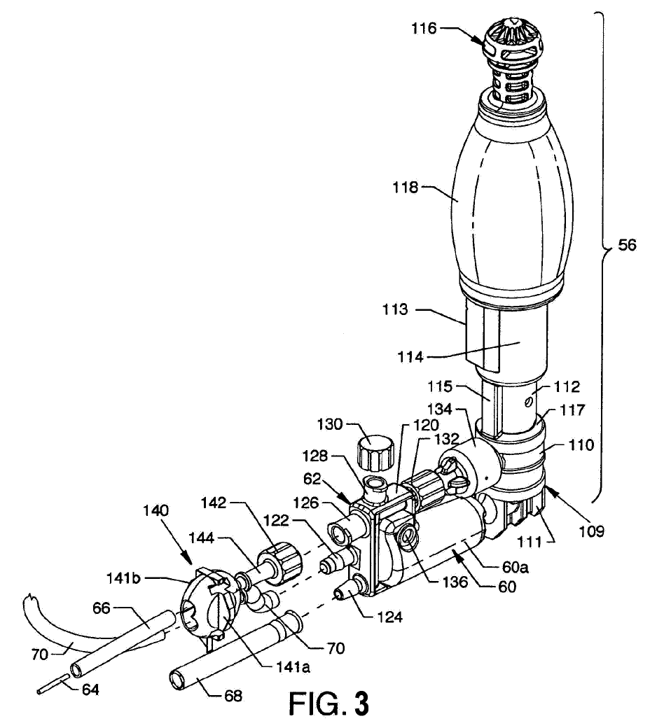

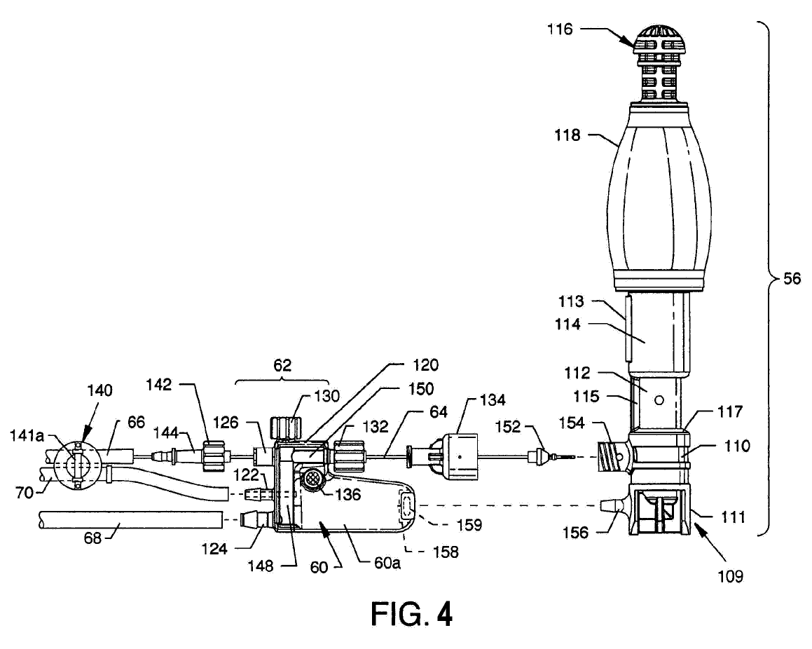

The pump/catheter assembly 14, which may be a disposable single-use device, is shown unattached from the console 12. The pump/catheter assembly 14 may include a pump 56 and a thrombectomy catheter 58. During use, a portion of the pump/catheter assembly 14 may be secured within a portion of the console 12. Other components included in the pump/catheter assembly 14 may include a bubble trap 60 attached to the pump 56, a connection manifold assembly 62 connected to the bubble trap 60, an effluent return tube 66 connected between the connection manifold assembly 62 and the thrombectomy catheter 58, a high-pressure fluid supply tube 64 (shown in FIGS. 3-4) attached between the output of the pump 56 and the thrombectomy catheter 58 which may be coaxially arranged inside the effluent return tube 66, a transition fixture 69 between the distal end of the effluent return tube 66 and the proximal end of the thrombectomy catheter 58, an effluent waste tube 68 connecting the effluent collection bag 28 to the connection manifold assembly 62, and a fluid supply tube 70 having a bag spike 71 connecting a fluid supply bag 72 (e.g., a saline bag) to the connection manifold assembly 62. The fluid supply tube 70 may be in fluid communication with the interior of the bubble trap 60 to provide fluid from the fluid supply bag 72 to the pump 56 and then to the thrombectomy catheter 58 through the high-pressure fluid supply tube 64.

In some examples, the effluent waste tube 68 may be coupled to a roller pump positioned within the console 12. In some examples, the effluent waste tube 68 may be coupled to a roller pump which may be positioned within the carriage assembly 22. It can be appreciated that the effluent waste tube 68 may be positioned and engaged with one or more rollers of the roller pump such that activation of the roller pump may compress consecutive segments of the effluent waste tube 68 (as the individual rollers of the roller pump engage the effluent waste tube 68), thereby propelling effluent liquid through the effluent waste tube 68 and into the effluent collection bag 28. In some examples, activation of the roller pump may occur via a foot switch connected to the console 12. For example, a user may depress the foot switch to activate the roller pump. It can be appreciated that when not activated, the roller pump may be in a closed configuration, preventing the flow of effluent and/or blood through the effluent waste tube 68. As discussed herein, activation of the roller pump may open up the effluent waste tubing, thereby opening up the effluent waste tube and permitting effluent including thrombus or other debris to flow into the effluent collection bag 28.

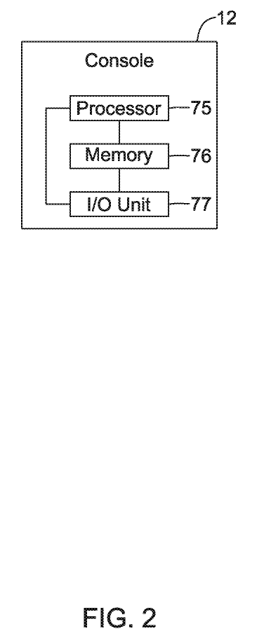

FIG. 2 illustrates that the console 12 may include, among other suitable components, one or more processors 75, memory 76, and an input/output (I/O) unit 77. The processor 75 of the console 12 may include a single processor or more than one processor (e.g., a first processor 75 providing data/instructions to the interface 32. The processor 75 may be configured to execute instructions, including instructions that may be loaded into the memory 76 and/or other suitable memory. Example processor components may include, but are not limited to, microprocessors, microcontrollers, multi-core processors, graphical processing units, digital signal processors, application specific integrated circuits (ASICs), field programmable gate arrays (FPGAs), discrete circuitry, and/or other suitable types of data processing devices. In some examples, the processor 75 of the console may be configured to execute program instructions. Program instructions may include, for example, firmware, microcode or application code that is executed by the processor 75, a microprocessor and/or microcontroller. The one or more processors 75 may be configured to each manage different functions. They may also be configured to concurrently perform the same functions (e.g., redundant system). Further yet, they may be configured such that a first processor 75 performs a given function and second processor 75 checks the result of the function of the first processor 75 for correctness (e.g., command-monitor system).

The memory 76 of the console 12 may include a single memory component or more than one memory component each working individually or with one another. Example types of memory may include random access memory (RAM), EEPROM, FLASH, suitable volatile storage devices, suitable non-volatile storage devices, persistent memory (e.g., read only memory (ROM), hard drive, optical disc memory, and/or other suitable persistent memory) and/or other suitable types of memory. The memory 76 may be or may include a non-transitory computer readable medium.

The I/O units 77 of the console 12 may include a single I/O component or more than one I/O component each working individually or with one another. Example I/O units 77 may be any type of communication port configured to communicate with other components of the thrombectomy system 10. Example types of I/O units 77 may include wired ports, wireless ports, radio frequency (RF) ports, Low-Energy Bluetooth ports, Bluetooth ports, Near-Field Communication (NFC) ports, HDMI ports, Wi-Fi ports, Ethernet ports, VGA ports, serial ports, parallel ports, component video ports, S-video ports, composite audio/video ports, DVI ports, USB ports, optical ports, and/or other suitable ports.

FIG. 3 is a partially exploded perspective view of several components of the pump/catheter assembly 14 generally including the pump 56, the bubble trap 60, the connection manifold assembly 62, etc. FIG. 3 illustrates that the pump 56 may center about a tubular body 112. Components are located about the lower region of the tubular body 112 and may include a base 109 having an upper portion 110 and a lower portion 111 both positioned about the lower region of the tubular body 112. An annular surface 117 is included at the top of the upper portion 110 of the base 109 for intimate contact with capture tabs of the carriage assembly 22 to contain the pump 56 within the carriage assembly 22. A top body 114, is positioned about the upper region of the tubular body 112. The base 109 and the top body 114, as well as a connecting panel 115, may be molded or otherwise suitably constructed to encompass the greater part of the tubular body 112, for example. A data plate 113 may also be included on the top body 114 for the inclusion of a barcode, an RFID tag, or other informational displays to determine operational parameters of the device.

FIG. 3 further illustrates that the pump 56 may include a hemispherically-shaped pump piston head 116 having a flexible boot 118 connected to and extending between the top body 114 and the pump piston head 116. In some instances, the geometrically configured lower portion 111 of the base 109 may serve as a mount for one end of the bubble trap 60. Additionally, the connection manifold assembly 62 may be secured directly to the other end of the bubble trap 60 and in some instances may include a bracket 120 to which is attached a vertically oriented tubular manifold 148 (shown in FIG. 4) having a plurality of ports attached or formed therethrough including a fluid (e.g., saline) inlet port 122, an effluent outlet port 124, a Luer style effluent return port 126, and/or an auxiliary port 128 and cap 130. Also shown are connectors 132 and 134 configured to extend between and engage the connection manifold assembly 62 and the upper portion 110 of the base 109. Additionally, a hydrophobic filter 136 may be included in an upper region of a bubble trap half 60a of the bubble trap 60. Another hydrophobic filter (not shown) may be disposed on the other bubble trap half that opposes the hydrophobic filter 136 on the bubble trap half 60a.

FIG. 3 further illustrates that a fixture 140, and components associated therewith, may assist in support and connection of the effluent return tube 66 to the effluent return port 126 by a connector 142 combined continuously with a connection tube 144, and also assists in support, passage and connection of the fluid supply tube 70 with the fluid inlet port 122. The fixture 140 includes outwardly extending vertically aligned and opposed tabs 141a and 141b which prevent the fixture 140 and associated effluent return tube 66 containing the high pressure saline supply tube 64 and the saline supply tube 70 from contacting a roller pump located in the carriage assembly 22.

FIG. 4 is a partially exploded side view of the elements of FIG. 3 illustrating the relationship of the pump 56, the bubble trap 60, the connection manifold assembly 62, and the fixture 140. Also shown is the vertically oriented tubular manifold 148 secured to the bracket 120. The effluent outlet port 124 may be connected to and in fluid communication with the lower interior of the tubular manifold 148. The effluent return port 126 may be connected to and in fluid communication with the upper interior of the tubular manifold 148. Also connecting to the tubular manifold 148 is a horizontally aligned passage port 150 and associated connector 132, each opposing the effluent return port 126. The passage port 150 may accommodate the high-pressure fluid supply tube 64 which extends distally through the lumen (not explicitly shown) of the passage port 150, the connector 132, the upper region of the tubular manifold 148, the effluent return port 126, the connector 142, the connection tube 144, and into and through the effluent return tube 66 to connect to the thrombectomy catheter 58 (FIG. 1). The proximal end of the high-pressure fluid supply tube 64 includes a high-pressure fitting 152 located near the proximal end of the high-pressure fluid supply tube 64 to facilitate connection of the high-pressure fluid supply tube 64 in fluid communication with the interior of the pump 56. The proximal end of the high-pressure fluid supply tube 64, which is the inlet to the high-pressure fluid supply tube 64, may include a plurality of very small holes (not shown) comprising a filter at the proximal end thereof. The connector 134, which may have internal and/or external threads, may be aligned over and about the high-pressure fluid supply tube 64 distal to the high-pressure fitting 152 and threadingly engage a threaded connection port 154 extending horizontally from the upper portion 110 of the base 109 of the pump 56. The connector 134 may be rotated to threadably engage the high-pressure fitting 152 with a corresponding mating threaded structure provided with the pump 56. A connector 132 may be utilized to engage the externally threaded end of the connector 134 to secure the connector 134, and thus the pump 56, to the connection manifold assembly 62 and to provide for fixation of the bubble trap 60 to the pump 56. In addition, direct connection and communication between the pump 56 and the bubble trap 60 is provided by a horizontally oriented pump saline inlet port 156 which engages a corresponding geometry receptor port 158 and seal 159 interior to one end of the bubble trap 60. The fluid inlet port 122 located on the bracket 120 may extend behind the tubular manifold 148 to communicate with the interior of the bubble trap 60 for fluid (e.g., saline) debubbling, whereby unpressurized fluid (e.g., saline) is made available for use by the pump 56.

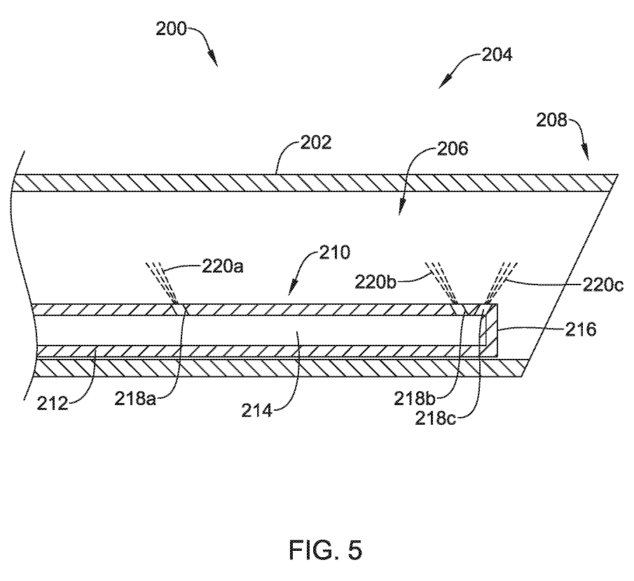

FIG. 5 is a cross-sectional view of a distal end region 204 of an example thrombectomy catheter 200. The thrombectomy catheter 200 shown in FIG. 5 may be one illustrative example of the thrombectomy catheter 58 described herein. The thrombectomy catheter 200 may include a tubular member or catheter body 202 extending from a proximal end region configured to remain outside the patient to a distal end region 204. The catheter body 202 may include a distal end 208. The catheter body 202 may be one illustrative example of, or be in fluid communication with, the effluent return tube 66 of the thrombectomy catheter 58 described herein. A lumen 206 defined within the catheter body 202 may be one illustrative example of, or be in fluid communication with, the lumen of the effluent return tube 66 of the thrombectomy catheter 58 described herein. Further, catheter body 202 may include one or more markers (e.g., radiopaque marker bands) disposed along the catheter body 202. Further yet, in some embodiments, the catheter body 202 may include one or more openings extending through a sidewall thereof, if desired.

The thrombectomy catheter 200 may further include a high-pressure fluid supply tube 210. The high-pressure fluid supply tube 210 may be one illustrative example of, or be in fluid communication with, the high-pressure fluid supply tube 64 of the thrombectomy catheter 58 described herein. The high-pressure fluid supply tube 210 may be disposed within and extend through the lumen 206 of the catheter body 202.

The high-pressure fluid supply tube 210 may include a supply tube wall 212 defining a lumen or fluid pathway 214 extending therethrough. In at least some instances, the high-pressure fluid supply tube 210 may have a closed distal end 216. Because of this, fluid may be able to pass distally through the fluid pathway 214 but does not exit the distal end. The high-pressure fluid supply tube 210 may extend along a length of the catheter body 202 with the distal end 216 located within the lumen 206 of the catheter body 202 proximal to the distal end 208 of the catheter body 202. A proximal end of the high-pressure fluid supply tube 210 may be in fluid communication with the pump 56 described herein, to provide high-pressure fluid to the fluid pathway 214 of the high-pressure fluid supply tube 210.

A plurality of jet orifices 218a-c (collectively, 218) may be defined along the supply tube wall 212. For example, the supply tube wall 212 may include two, three, four, five, six, or more jet orifices 218. The jet orifices 218 may be spaced along the supply tube wall 212 at any desired intervals. For example, each of the jet orifices 218 may be equidistantly spaced from adjacent jet orifices 218 along the length of the supply tube wall 212. In other instances, the jet orifices 218 may be arranged such that the spacing between adjacent jet orifices 218 near the distal end of the supply tube wall 212 is closer than the spacing between adjacent jet orifices 218 near the proximal end of the supply tube wall 212. For instance, the spacing between the orifices 218 may gradually increase as you move proximally along the length of the shaft, or the spacing may increase in a stepwise configuration. In some instances, some or all of the jet orifices 218 may be axially aligned along the supply tube wall 212. In other instances, one or more of the jet orifices 218 may be circumferentially offset from one another about the supply tube wall 212. A number of patterns are contemplated including a helical pattern, a pattern where no two jet orifices 218 are disposed at the same axial location, a regular pattern including two or more jet orifices 218 disposed at the same axial location, an irregular pattern (where some of the jet orifices 218 may or may not be disposed at the same axial location), etc.

The jet orifices 218 may be formed using a suitable method such as electron discharge machining, etching, cutting (e.g., including laser cutting), or the like. In some instances, one or more of the jet orifices 218 may have a substantially round shape. In other instances, one or more of the jet orifices 218 may have a substantially non-round shape (e.g., oval, polygonal, irregular, etc.). In some instances, the jet orifices 218 may be beveled or otherwise include a beveled surface. It is contemplated that a size and/or a shape of the jet orifices 218 may be varied to vary the velocity of the fluid exiting the jet orifices. For example, decreasing the size of the jet orifices 218 may increase the velocity of the fluid exiting the jet orifices 218. In some embodiments, the size of the jet orifices 218 may be varied based on the pressure capacity of the thrombectomy system, the number of jet orifices, the dimensions of the high-pressure fluid supply tube 210 (e.g., length, wall thickness, inner diameter, etc.), and/or combinations thereof. In some examples, the jet orifices 218 may have a cross-sectional dimension in the range of about 0.0005 inches (0.0127 millimeters (mm)) to about 0.0030 inches (0.0762 mm), or about 0.0010 inches (0.0254 mm) to about 0.0025 inches (0.0635 mm), or about 0.0015 inches (0.0381 mm) to about 0.0020 inches (0.0508 mm).

Infusion of fluid through the lumen 214 of the supply tube wall 212 may result in fluid being jetted through the jet orifices 218 and the generation of a proximally-directed aspiration force. At least some of the jet orifices 218a-b may be angled in a proximal direction or otherwise designed to infuse fluid (e.g., a motive fluid, a liquid, a gas or air, steam, a fluid with particles disposed therein, or the like) through the jet orifices 218a-b and into the lumen 206 of the catheter body 202 in a generally proximal direction as depicted by lines 220a-b representing motive jetted fluid projecting generally proximally from the jet orifices 218a-b. For example, each of the jet orifices 218a-b may be arranged at an acute angle to the longitudinal axis of the supply tube wall 212 such that the jet orifices 218a-b angle in a proximal direction.

In some embodiments, one or more of the jet orifices 218c may be designed to infuse fluid (e.g., a motive fluid, a liquid, a gas or air, steam, a fluid with particles disposed therein, or the like) through the jet orifice(s) 218c and into the lumen 206 of the catheter body 202 in a generally distal direction as depicted by lines 220c representing motive jetted fluid projecting generally distally from the jet orifice 218c. For example, the jet orifice 218c may be arranged at an oblique angle to the longitudinal axis of the supply tube wall 212 such that the jet orifice 218c angles in a distal direction. It is contemplated that an angle of the jet orifices 218 and thus the motive jetted fluid 220 may be varied to adjust the velocity of the fluid exiting the jet orifices 218. As further described herein, the supply tube wall 212 may include one or more, or a plurality of proximally oriented or directed jet orifices 218a, 218b (i.e., jet orifices configured to direct fluid infused through the lumen 214 of the supply tube wall 212 in a proximal direction) and the supply tube wall 212 may include one or more, or a plurality of distally oriented or directed jet orifices 218c (i.e., jet orifices configured to direct fluid infused through the lumen 214 of the supply tube wall 212 in a distal direction). In some examples, the distally projecting jet orifice 218c may be axially aligned with one or more of the proximally projecting jet orifices 218a-b. In other examples, the distally projecting jet orifice 218c may be circumferentially offset from one or more of the proximally projecting jet orifices 218a-b. For example, the distally projecting jet orifice 218c may be circumferentially offset from one or more of the proximally projecting jet orifices 218a-b by in the range of about 10° to about 350° or about 45° to about 135°.

In some examples, the distally projecting jet orifice 218c may be the distalmost jet orifice, with the proximally projecting jet orifices 218a-b positioned proximal of the distally projecting jet orifice 218c. However, in other examples, the distally projecting jet orifice 218c may be positioned proximal to at least one proximally projecting jet orifice 218a-b. While the supply tube wall 212 is illustrated as including only a single distally projecting jet orifice 218c, the supply tube wall 212 may include more than one distally projecting jet orifice, as desired. When more than one distally projecting jet orifice 218c is provided, the distally projecting jet orifices may be positioned at differing axial and/or circumferential locations from one another or similar axial and/or circumferential locations as one another, as desired. The distally projecting jet orifice(s) 218c may break up particles as they are drawn into the lumen 206 of the catheter body 202 while the proximally projecting jet orifices 218a-b may move particles proximally along the catheter body 202.

The performance of the thrombectomy catheter 200 and the high-pressure fluid supply tube 210 may be directly related to the velocity of the motive jetted fluid 220 exiting the jet orifices 218 and the shear-induced turbulent flux created by the jetted motive fluid 220. For example, the more powerful the jetted motive fluid 220, the higher the aspiration rates may be. It is further contemplated that the performance of the jet-powered aspiration catheter 200 may be directly related to the speed at which the clot can be entrained into the catheter 200, macerated, and removed from the body. Any clogging that occurs within the catheter body 202 may reduce or completely stop the removal of the clot.

In some instances, the jet orifices 218 may be oriented at an angle relative to the longitudinal axis of the supply tube wall 212. For example, the proximally projecting jet orifices 218a-b may be oriented at an oblique (e.g., acute) angle relative to the longitudinal axis of the supply tube wall 212 and/or oriented at an angle greater than zero degrees and less than ninety degrees relative to the longitudinal axis of the supply tube wall 212. It is contemplated that a distally projecting jet orifice 218c may be oriented at an oblique (e.g., obtuse) angle relative to the longitudinal axis of the supply tube wall 212 and/or oriented at an angle greater than 90 degrees and less than 180 degrees relative to the longitudinal axis of the supply tube wall 212. In other instances, the jet orifices 218 may be oriented perpendicular to the longitudinal axis of the supply tube wall 212 (e.g., at an angle of about 90 degrees relative to the longitudinal axis of the supply tube wall 212). The angle may or may not be the same for all the jet orifices 218.

In at least some instances, the jet orifices 218 may be understood as being arranged in series. In other words, the jet orifices 218 may be arranged such that adjacent jet orifices 218 are spaced longitudinally apart at various locations along the longitudinal axis of the supply tube wall 212. For example, the jet orifices 218 may be uniformly or non-uniformly spaced along of a length of the supply tube wall 212. This may position the jet orifices 218 at axially spaced apart locations within the catheter body 202 and along the length thereof. For example, the jet orifices 218 may be spaced along an entire length of the supply tube wall 212 and correspondingly along an entire length of the catheter body 202, or portions thereof, as desired. In some examples, the jet orifices 218 may be spaced at intervals in the range of every 5 inches (12.7 centimeters (cm)) to every 15 inches (38.1 cm), or in the range of every 6 inches (15.2 cm) to every 12 inches (30.5 cm) along a length of the supply tube wall 212. In other instances, the spacing between the jet orifices 218 may be less than every 5 inches (12.7 cm) or greater than every 15 inches (38.1 cm). Accordingly, motive fluid leaves via the jet orifices 218 forming a jetted motive fluid 220a-c (collectively, 220).

In some instances, entrainment material may enter the distal opening of the catheter body 202 and then may be urged proximally by momentum transfer. As the mixture of the jetted motive fluid 220 and entrainment material migrates proximally, the material may sequentially approach a number of the jet orifices 218. Upon interaction with the jetted motive fluid 220 from each individual jet orifice 218, the momentum in the entrainment material mixture may increase, and the thrombogenic material may more readily flow proximally through the catheter body 202 for removal. The increase in momentum may allow for the catheter body 202 to be used without a second or outflow orifice (e.g., positioned proximally of the distal end 208). Alternatively, some of the entrapped thrombogenic material may exit the catheter body 202 through a second orifice (not shown), e.g., in a sidewall of the catheter body 202, positioned proximal to the distal end 208, recirculate to the distal end 208 (e.g., one or more times), and then move proximally through the lumen 206 of the catheter body 202.

As discussed herein, cycling of the high pressure pump 56 may provide high-pressure fluid to the fluid pathway 214 of the high-pressure fluid supply tube 210. For example, during the upstroke of the piston of the pump 56, saline fluid may be drawn into the pump 56 through the fluid supply line 70 and the low-pressure inlet port 122. The fluid may further pass through a check ball valve and into an inner chamber of the pump 56. On the downstroke of the piston of the pump 56, the saline fluid (which had been drawn into the chamber of the pump 56 on the upstroke) may be forced out of the chamber of the pump 56 and into the high pressure fluid supply tube 64 (depicted as high pressure fluid supply tube 210 in FIG. 5). As discussed herein, the high pressure motive fluid may flow through the lumen 214 of the high pressure supply tube 210 and pass through each individual jet orifice 218. As discussed herein, after passing through each individual jet orifice 218, the high pressure fluid may collect and transport thrombogenic material proximally through the lumen 206 of the catheter body 202 for removal. As discussed herein, the effluent fluid may be transported proximally via the effluent return tube 66 and into the effluent waste tube 68, whereby activation of the roller pump may open up the effluent waste tube 68 and permit the effluent fluid to be transported into the effluent collection bag 28.

It can be further appreciated that between the upstroke/downstroke cycling of the piston of the pump 56 and the opening/closing of the effluent waste tube 68 via actuation of the roller pump, a static pressure differential may develop within the lumen 206 of the catheter body 202. For example, during the downstroke of the piston of the pump 56, pressure may build up within the lumen 206 of the catheter body 202. This increase in pressure may occur because the proximal volumetric flowrate of fluid through the lumen 206 of the catheter body 202 may exceed the volumetric outflow of fluid through the effluent waste tube 68. For example, in some instances, the cross-sectional opening of the lumen of the effluent waste tube 68 may close, thereby preventing the transport of effluent fluid through the effluent waste tube 68 and into the effluent waste collection bag 28 without a buildup of pressure occurring within the lumen 206 of the catheter body 202.

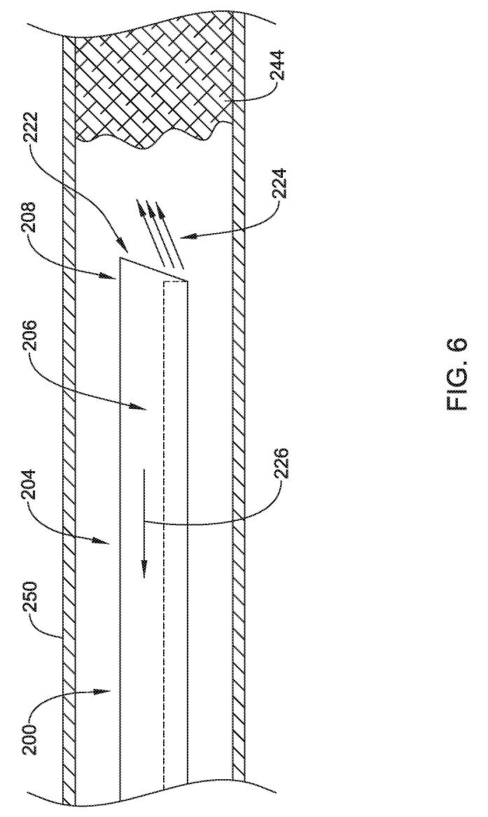

FIG. 6 illustrates that if the pressure buildup in the lumen 206 of the catheter body 202 is not released during the cycling of the piston of the pump 56, the excess pressure buildup in the lumen 206 of the catheter body 202 may result in motive fluid being ejected distally out of the opening 222 in the distal end 208 of the catheter 200. For example, FIG. 6 illustrates that the catheter 200 is positioned within a body lumen 250. FIG. 6 illustrates the distal end 208 of the catheter 200 positioned proximal to thrombogenic material 244 in the body lumen 250. Additionally, FIG. 6 illustrates some effluent fluid flowing in a proximal direction within the lumen 206 after having passed through the jets 218 (shown in FIG. 5). This proximal high pressure fluid flow may correlate with and result from the downstroke of the piston of the pump 56. As discussed herein, if fluid pressure build up during the downstroke of the piston of the pump 56 is not released during the cycling of the piston, excess fluid may be ejected out of the distal end 208 of the catheter 200. As discussed herein, FIG. 6 illustrates high pressure fluid being ejected out of the opening 222 in the distal end 208 of the catheter 200 as shown by the directional arrows 224 in FIG. 6.

It can be appreciated that the thrombectomy system 10 may include one or more features configured to rapidly relieve the pressure buildup in the lumen 206 of the catheter 200. For example, the thrombectomy system 10 may be configured to include an effluent pressure relief valve 74 positioned along a component of the thrombectomy system 10. For example, the pressure relief valve 74 may be positioned along the connection manifold assembly 62, effluent return tube 66, the effluent waste tube 68, the transition fixture 69 or any other suitable location within the thrombectomy system 10. In other examples, manipulating the rotation speed and/or the opening of the roller pump may be utilized to alleviate the buildup of excess pressure in the lumen 206 of the catheter 200. In other words, the roller pump may be utilized as a pressure relief valve, whereby a properly timed opening of the roller pump may rapidly relieve the pressure buildup in the lumen 206 of the catheter 200. It can be appreciated that the roller pump may be configured to open and close (and thereby rapidly relieve the pressure buildup in the lumen 206 of the catheter 200) even when not rotating. In other examples, the roller pump may be utilized as a pressure relief valve, whereby manipulation of the rotation speed of the roller pump may rapidly relieve the pressure buildup in the lumen 206 of the catheter 200. In yet other examples, the effluent pressure relief valve 74 may be used in conjunction with the roller pump to rapidly relieve the pressure buildup in the lumen 206 of the catheter 200.

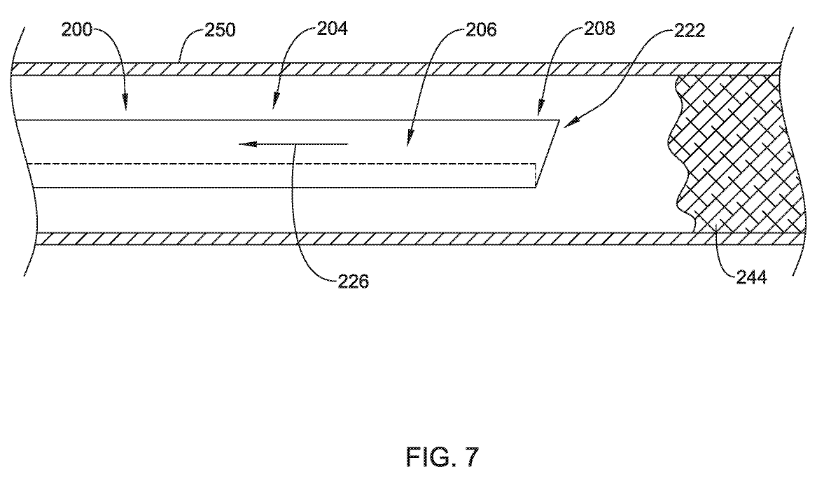

As discussed herein, the timing the opening of a relief valve positioned along the effluent fluid pathway during the piston upstroke/downstroke cycle, for example, and/or manipulation of the rotation speed of the roller pump and/or the opening of the roller pump, may rapidly relieve excess pressure buildup in the lumen 206 of the catheter 200 and thereby prevent the ejection of high pressure fluid out of the opening 222 in the distal end 208 of the catheter. For example, FIG. 7 illustrates the catheter 200 shown in FIG. 6 whereby the ejection of high pressure fluid out of the opening 222 in the distal end 208 of the catheter has been prevented by the opening of an example relief valve 74 during the piston upstroke/downstroke cycle, for example, and/or manipulating the rotation speed of the roller pump and/or the opening of the roller pump while the roller pump is not rotating.

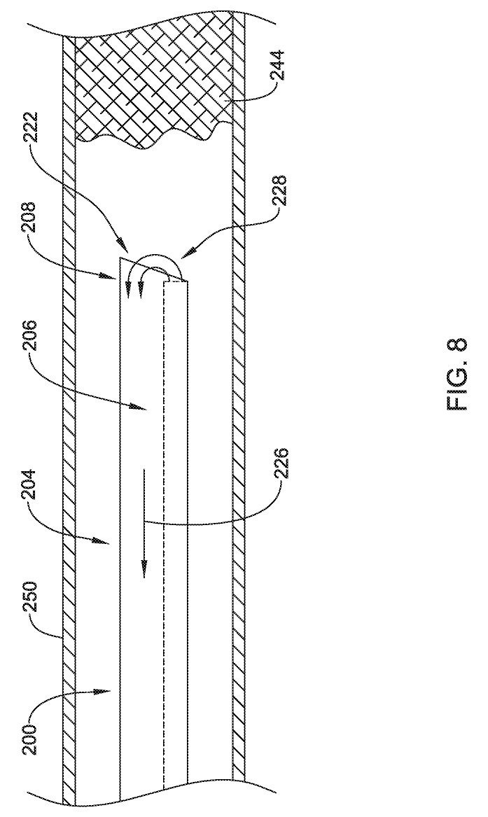

Further, in some instances it may be desirable to time the opening of the pressure relief valve 74 during the piston upstroke/downstroke cycle and/or coordinate the rotation speed of the roller pump to create a negative pressure within the lumen 206 of the catheter 200. It can be appreciated that creating a negative pressure within the lumen 206 of the catheter 200 may create a localized zone of an inwardly-directed, short-interval, vacuum pressure which may increase and/or enhance the vacuum effect created by the jetted motive fluid 220 (shown in FIG. 5). This increased vacuum effect created by the inwardly-directed, short-interval, vacuum pressure within the lumen 206 of the catheter 200 may help to macerate and entrain thrombus into the lumen 206 of the catheter 200. For example, FIG. 8 illustrates a localized zone of an inwardly-directed, short-interval burst of vacuum pressure which creates a vacuum effect and draws fluid into the distal end 208 of the catheter 200 (as shown by the directional arrows 228 in FIG. 8). As described herein, this increased vacuum effect created by the inwardly-directed, short-interval, burst of vacuum pressure at the distal end 208 of the catheter 200 may help to macerate and entrain the thrombus 244 into the lumen 206 of the catheter 200, whereby it is removed from the patient and into the effluent collection bag 22 via the effluent return tube 66 and the effluent waste tube 68.

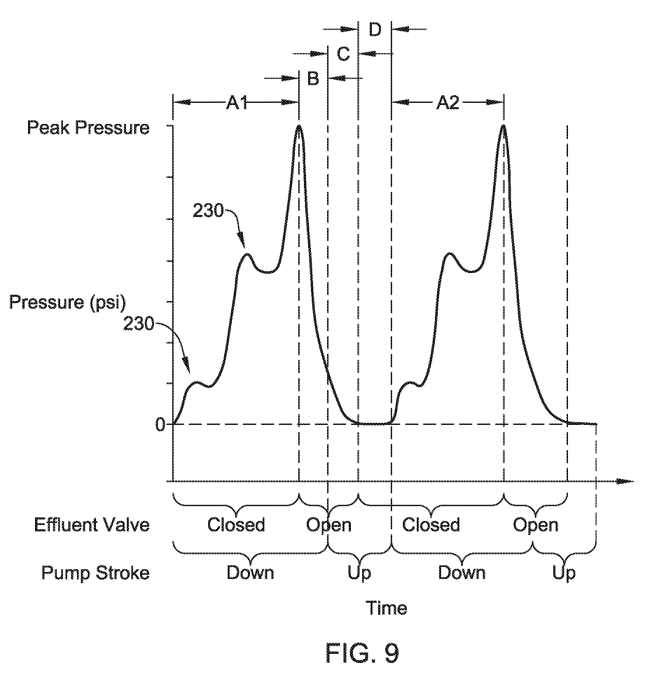

FIG. 9 is graphical representation of the pressure within the lumen 206 at the distal end 208 of the catheter 200 over two consecutive pressure cycles, whereby each individual pressure cycle occurs over a time period that spans a downstroke and upstroke of the piston of the pump 56. For simplicity, FIG. 9 will be described as representing the thrombectomy system 10 which includes the effluent pressure relief valve 74 configured to be opened at a specific time to create a localized zone of an inwardly-directed, short-interval burst of pressure within the lumen 206 of the catheter 200, as described herein. However, as described herein, other example thrombectomy systems may include alternative methods of creating a localized zone of an inwardly-directed, short-interval burst of pressure within the lumen 206 of the catheter 200, such as the timed opening/closing of a roller pump (while not rotating, for example), and/or manipulating the rotation speed of the roller pump, and/or opening a roller pump used in conjunction with the effluent pressure relief valve 74.

FIG. 9 illustrates a time interval “A1” in which the piston of the pump 56 is beginning a downstroke. The time interval A1 may represent the period during the downstroke of the piston in which an effluent pressure relief valve 74 may be closed. In some examples, the effluent valve may remain closed over 65 to 100 percent of the downstroke time period. For example, if the downstroke time period is equal to 1 second, the effluent pressure relief valve 74 may remain closed between 0.65 second to 1 second. In other words, as the piston begins its downstroke (illustrated in FIG. 9 as the sum of time interval A1 and time interval B), the effluent pressure relief valve 74 may be closed, whereby after 65 to 100 percent of the total downstroke time interval has passed (illustrated in FIG. 9 as the interval A1), the effluent pressure relief valve 74 may open. FIG. 9 illustrates that the effluent pressure relief valve 74 may open before the downstroke of the piston has been completed.

Further, it can be appreciated that pressure may increase in the lumen 206 of the catheter 200 during the downstroke of the piston because the volume of high pressure fluid being injected into the high pressure fluid supply tube 64 may not be vacated through the effluent return tube 66 quickly enough without an increase in pressure. FIG. 9 illustrates that the pressure may increase in the lumen 206 of the catheter 200 from approximately 0 psi (pressure per square inch) to a “peak pressure,” which, in some examples, may range from about 14 psi (pounds per square inch) (96.53 kilopascal (kPa)) to 26 psi (179.26 kPa), or about 16 psi (110.32 kPa) to about 24 psi (165.47 kPa), or about 18 psi (124.11 kPa) to about 22 psi (151.68 kPa), or about 20 psi (137.90 kPa). In some examples, the peak pressure may be sensed by one or more components of the thrombectomy system 10. For example, the effluent pressure relief valve 74 may be coupled to a pressure sensor and/or the processor 75, whereby the pressure sensor and/or processor 75 is configured to sense a peak pressure value within the lumen of the catheter body, and whereby the sensor and/or processor 75 is configured to automatically actuate the effluent pressure relief valve 74 in response to sensing a threshold or targeted peak pressure value.

Further, FIG. 9 illustrates one or more pressure “dips” 230 during the interval A1. It can be appreciated that each dip 230 may correspond to the opening of the effluent waste tube 68 via activation (e.g., rotation) of the roller pump. In other words, rotation of the roller pump may open the effluent waste tube 68 at set intervals, which is depicted in the graph of FIG. 9 by the sequential pressure dips 230. Further, it can be appreciated that the sequential pressure dips 230 may also be generated via the opening/closing of the roller pump, whereby the opening/closing of the roller pump may occur when the roller pump is not spinning. Further yet, it can be appreciated that the sequential pressure dips 230 may also be generated via the incorporation of a high frequency pulsation on/off valve which may be positioned along the thrombectomy system 10.

In some examples, the interval A1 may be about 0.100 seconds to about 1.0 seconds, or about 0.125 seconds to about 0.850 seconds, or about 0.170 seconds to about 0.775 seconds.

FIG. 9 further illustrates a time interval “B” in which the piston of the pump 56 remains in the downstroke but the effluent pressure relief valve 74 opens (illustrated in FIG. 9 as the pump downstroke time period minus the effluent valve close time period). As discussed herein, the opening of the effluent pressure relief valve 74 may result in a rapid decrease in pressure, which is depicted in the time interval B of the graph of FIG. 9. As discussed herein, this rapid decrease in pressure may initiate the localized zone of an inwardly-directed, short-interval vacuum pressure at the distal end 208 of the catheter 200. It can be appreciated that the point at which the pressure relief valve is opened may define the peak pressure within the lumen 206 at the distal end 208 of the catheter 200. Additionally, in some examples, it may be desirable to coordinate the opening of the effluent pressure relief valve 74 with the opening of the effluent waste tube 68. It can be appreciated that coordinating the timing of opening the effluent pressure relief valve 74 with the opening of the effluent waste tube 68 by the roller pump may create an even greater drop in pressure over time verses opening the pressure relief valve alone. It can be further appreciated that the effluent pressure relief valve 74 may be coupled to a vacuum source, whereby coordinating the opening of the effluent pressure relief valve 74 with activation of the vacuum source (e.g., exposing the effluent pressure relief valve 74 to a vacuum source) may create an even greater drop in pressure over time verses opening the pressure relief valve alone.

In some examples, the interval B may be about 0.008 seconds to about 0.350 seconds, or about 0.010 seconds to about 0.275 seconds, or about 0.012 seconds to about 0.245 seconds. It can be appreciated that the interval B is exemplary and may vary depending on the specific configuration of the thrombectomy system. The time interval B may be adjusted to assure that the pressure buildup in the lumen 206 of the catheter 200 is fully dissipated. For example, the configuration of the catheter components of the thrombectomy system 10 may require the time interval B to be adjusted (e.g., lengthened). For example, the overall length of the catheter 200 and/or the diameter of the lumen 206 of the catheter may influence the length of time interval B. For example, a relatively longer catheter 200 or smaller catheter lumen diameter 206 may require a relatively longer B interval while a relatively shorter catheter 200 or larger catheter lumen diameter 206 may allow a shorter B interval.

FIG. 9 further illustrates a time interval “C” in which the effluent pressure relief valve 74 remains open but the piston changes direction from the downstroke to the upstroke. As discussed herein, during the upstroke, the piston may begin to draw fluid into the piston chamber (versus the downstroke in which the piston is injecting fluid into the high pressure fluid supply tube 64). It can be appreciated from FIG. 9 that the pressure during the interval C may continue to decrease to 0 psi or ambient pressure. Further, in other examples, the pressure may fall below 0 psi, thereby creating a negative pressure within the lumen 206 at the distal end 208 of the catheter 200. In some examples, the time interval C may be approximately 50 percent of the pump upstroke time interval.

In some examples, the interval C may be about 0.050 seconds to about 0.150 seconds, or about 0.065 seconds to about 0.135 seconds, or about 0.078 seconds to about 0.118 seconds.

FIG. 9 further illustrates a time interval “D” in which the piston of the pump 56 remains in the upstroke but the effluent pressure relief valve 74 closes. It can be appreciated that pressure may increase in the distal end 208 of the catheter 200 during the upstroke of the piston because the pressure relief valve has closed, thereby creating a pressure differential in the distal end 208 of the catheter 200, as described herein. In some examples, the time interval D may be approximately 50 percent of the pump upstroke time interval. In some examples, the time interval D may be approximately equal to the pump upstroke time interval minus the time interval C. In yet other examples, the time interval D may be approximately equal to the time interval C. In some examples, the interval D may be about 0.050 seconds to about 0.150 seconds, or about 0.065 seconds to about 0.135 seconds, or about 0.078 seconds to about 0.118 seconds.

It can be further appreciated that the time interval D immediately precedes a time interval A2 of a second pressure cycle. As described herein, the second time interval A2 may begin when the piston changes direction from the upstroke to the downstroke. Accordingly, the increasing pressure illustrated in the time interval D leads directly into the increasing pressure of the second time interval A2. It can be appreciated that this pressure cycle then repeats through time intervals B, C and D to complete the second pressure cycle. The materials that can be used for the various components of the thrombectomy catheter, pump/catheter assembly, and/or other devices disclosed herein may include those commonly associated with medical devices. For simplicity purposes, the following discussion refers to the pump/catheter assembly and its related components. However, this is not intended to limit the devices and methods described herein, as the discussion may be applied to other similar devices, tubular members and/or components of tubular members or devices disclosed herein.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the invention is, of course, defined in the language in which the appended claims are expressed.

What is claimed is:

1. A thrombectomy system, comprising:
a thrombectomy catheter including a distal end region and a lumen extending therein;
a fluid supply tube extending within the lumen of the thrombectomy catheter, wherein the fluid supply tube includes one or more fluid supply jets; and
a pressure relief member coupled to the thrombectomy catheter, wherein the pressure relief member is configured to control effluent fluid flow within the lumen of the thrombectomy catheter;
wherein actuation of the pressure relief member is configured to relieve pressure in the lumen of the thrombectomy catheter.

2. The system of claim 1, wherein in the pressure relief member includes a pressure relief valve.

3. The system of claim 2, wherein actuation of the pressure relief valve from a closed configuration to an open configuration is configured to rapidly relieve pressure in the lumen of the distal end region of the thrombectomy catheter.

4. The system of claim 3, wherein rapidly relieving pressure in the lumen of the distal end region of the thrombectomy catheter generates a vacuum force directed inwardly into the lumen of the thrombectomy catheter.

5. The system of claim 4, wherein the vacuum force is configured to macerate a thrombus positioned distal to the distal end of the thrombectomy catheter.

6. The system of claim 1, wherein actuation of the pressure relief member is configured to decrease the pressure within the lumen of the distal end region of the thrombectomy catheter from a peak pressure between 18 psi (124.11 kPa) and 22 psi (151.68 kPa) to a pressure of 0 psi (0 kPa).

7. The system of claim 6, wherein relieving the pressure from the peak pressure between 18 psi (124.11 kPa) and 22 psi (151.68 kPa) to the pressure of 0 psi (0 kPa) occurs over a time period of 0.075 seconds to 0.4 seconds.

8. The system of claim 1, wherein actuation of the pressure relief member is configured to decrease the pressure within the lumen of the distal end region of the thrombectomy catheter from a peak pressure between 18 psi (124.11 kPa) and 22 psi (151.68 kPa) to a pressure below 0 psi (0 kPa).

9. The system of claim 1, wherein the proximal end region of the thrombectomy catheter is coupled to a fluid pump, and wherein the fluid pump is configured to cycle between a downstroke and an upstroke, and wherein actuation of the pressure relief member occurs during the downstroke.

10. The system of claim 9, wherein the pressure relief member includes a pressure relief valve, and wherein the downstroke occurs between a first time and a second time, and wherein actuation of the pressure relief valve occurs before the second time.

11. The system of claim 10, wherein actuation of the pressure relief valve opens the pressure relief valve, and wherein the pressure relief valve remains open over a third time period between 0.075 seconds to 0.4 seconds.

12. The system of claim 1, wherein the pressure relief member includes a roller pump.

13. The system of claim 12, wherein the roller pump is configured to cycle between an open configuration and a closed configuration, and wherein cycling the roller pump is configured to rapidly relieve pressure in the lumen of the distal end region of the thrombectomy catheter.

14. The system of claim 13, wherein the roller pump is configured to sense a peak pressure in the lumen of the thrombectomy catheter, and wherein the roller pump is configured to cycle between the open configuration and the closed configuration to rapidly relieve pressure in the lumen of the distal end region of the thrombectomy catheter based on the sensing of the peak pressure.

15. A thrombectomy system, comprising:
a processor coupled to a fluid pump and a thrombectomy catheter, wherein the thrombectomy catheter includes a distal end region and a lumen extending therein;
a fluid supply tube extending within the lumen of the thrombectomy catheter, wherein the fluid supply tube includes one or more fluid supply jets; and
a pressure relief member coupled to the processor and the thrombectomy catheter, wherein the pressure relief member is configured to control effluent fluid flow within the lumen of the thrombectomy catheter;
wherein actuation of the pressure relief member is configured to relieve pressure in the distal end region of the thrombectomy catheter.

16. The system of claim 15, wherein in the pressure relief member includes a pressure relief valve configured to rapidly relieve pressure in the lumen of the distal end region of the thrombectomy catheter.

17. The system of claim 16, wherein the processor is configured to sense a peak pressure value within the lumen of the thrombectomy catheter, and wherein the processor is configured to automatically actuate the pressure relief valve in response to sensing the peak pressure value.

18. The system of claim 17, wherein the peak pressure value is between 18 psi (124.11 kPa) and 22 psi (151.68 kPa).

19. The system of claim 18, wherein the processor is configured to actuate the pressure relief valve over a time period between 0.075 seconds to 0.4 seconds.

20. A thrombectomy system, comprising:
a thrombectomy catheter including a distal end region and a lumen extending therein;
a fluid supply tube extending within the lumen of the thrombectomy catheter, wherein the fluid supply tube includes one or more fluid supply jets;
a pressure relief valve coupled to the thrombectomy catheter, wherein the pressure relief valve is configured to control effluent fluid flow within the lumen of the thrombectomy catheter; and
a roller pump coupled to the thrombectomy catheter, wherein the roller pump is configured to control effluent fluid flow within the lumen of the thrombectomy catheter;
wherein actuation of the pressure relief valve, the roller pump or both the pressure relief valve and the roller pump is configured to relieve pressure in the lumen of the thrombectomy catheter.
